(19) United States
(12) Patent Application Publication
(10) Pub. No.: US 2024/0195938 A1
(43) Pub. Date: Jun. 13, 2024

(54) METHOD AND SYSTEM FOR PROVIDING AN ON-LINE CONFERENCE INCLUDING A MEDICAL IMAGE

(71) Applicant: Medical iSight (UK) Ltd, London (GB)

(72) Inventor: Robert Merrifield, Enfield (GB)

(21) Appl. No.: 18/533,500

(22) Filed: Dec. 8, 2023

(30) Foreign Application Priority Data
Dec. 9, 2022 (GB) 2218539.1

(51) Int. Cl. H04N 7/15; G16H 30/20; G16H 30/40

(52) U.S. Cl. CPC H04N 7/15; G16H 30/20; G16H 30/40

(57) ABSTRACT

A computing system comprises a local store for containing at least one medical image and a display for displaying a medical image from the local store, and an audio system for receiving audio input from a user of the computing system; such audio input may relate to the medical image, a graphics system for the user to perform multiple different image manipulations of the medical image using graphics commands, and conferencing software to perform real-time communications with one or more other computing systems as part of an on-line conference, which includes: sharing the audio input from the user with the one or more other computing systems. Performing real-time communications further includes sharing the graphics commands with the one or more other computing systems to allow the one or more other computing systems to locally perform the different image manipulations as part of the on-line conference.

### FIELD

The present application relates to a method and system for providing an on-line conference involving at least one medical image.

### BACKGROUND

There is extensive use of imaging in the medical field, and a wide range of imaging modalities are available to a clinician including, without limitation, X-ray, CT scans, MRI scans, ultrasound, endoscopy and so on. In some cases, a patient may be imaged using multiple imaging modalities. Medical images may be acquired in two spatial dimensions (2D) or 3 spatial dimensions (3D). Also, some medical imaging such as endoscopy may span the temporal dimension to provide a sequence of images, e.g. in the form of a video. Such medical imaging can generate very large data sets—e.g. a 3D scanning session may generate 1 GByte or more of imaging data.

There are various ways in which such images may be utilised (in addition to direct visual inspection of the original image). In some cases, an image may be segmented to identify anatomical components within the image. This can lead to the generation of a model of patient anatomy. For present purposes this model can also be regarded as a form of medical imaging, since it may be displayed and visually inspected in a similar manner to the original image. Furthermore, multiple images of different modalities may be spatially registered with one another and then combined to produce a composite medical image.

A common practice, especially in complex cases, is for a team of multiple clinicians, e.g. with different specialties, to review information including medical images relating to a patient. Such a review typically seeks to determine and understand the medical condition of a patient and also to select an appropriate course of treatment.

Such reviews have typically been held as in-person meetings. However, with the onset of COVID and new ways of working, there has been an increasing use of video conferencing to implement such meetings to help reduce the spread of virus that may arise from an in-person meeting. Such video conferencing is often performed using standard platforms such as Zoom and Teams which are readily installed on personal computers without requiring any additional hardware. Such platforms support both audio and video transmissions between the different participants. In addition, a first participant is able to share an application with other participants, in the sense that the output from this application is made visible to the other participants. In the context of performing a medical review, this facility may be used by the first participant to share medical images with the other participants. Note that sharing an application in this manner does not allow the other participants to directly interact with that application, e.g. by making their own input into the application; rather they only see the output resulting from the actions and inputs of the first participant with respect to the application.

Video conferencing may also be utilised in other medical contexts, in addition to performing case reviews such as described above. For example, in a related field of tele-medicine, practitioners at one location, such as a major hospital with expertise in certain types of medicine, may provide on-line guidance to medical practitioners who do not have such expertise, but are directly attending a patient, perhaps in a relatively remote location. There is some overlap between performing a case review and performing telemedicine, although the latter may also include providing real-time guidance concerning an ongoing medical intervention (such as surgery) which is currently being performed.

One difficulty faced by the use of video conferencing in a medical context is that standard video conferencing platforms are primarily designed to provide rapid sharing of relatively low volumes of data. In contrast, such platforms are not primarily designed for sharing large data sets, such as 1 GByte medical images.

When handling such a large image data set, a video conferencing system may be designed to communicate first a low resolution version of an image which therefore is reduced in size—i.e. it comprises fewer bytes than the original version of the image. This reduced image can be transmitted more quickly to the recipients and displayed more quickly to these recipients. Subsequently, and over a longer period, the image may be transmitted at high resolution to provide the full detail of the original image.

However, this approach does not work well in the context of medical imaging, since it may not be apparent to a user that a displayed image is missing some high resolution detail. Therefore, a clinician may come to an erroneous (or less satisfactory) interpretation of an image because of this missing detail (of which the clinician may not be aware). There is also the possibility of confusion between two different clinicians. For example, one clinician may have the full resolution image displayed with all the details, while another clinician might be seeing a lower resolution version of the same image. This may lead to some confusion or discrepancy between the two clinicians, which might prove detrimental to the diagnosis or treatment of a patient.

Problems may also occur without the use of low and high resolution images. For example, a participant in a video conference may make audio (oral) comments about the displayed image. Such audio contents may be transmitted quickly to the other participants with little delay (latency) because a real-time audio signal can be transmitted using a relatively low bandwidth, e.g. a few Kbytes per second. In contrast, at this bit rate it would take many hours to transmit a 1 GByte medical image dataset. Even at a higher bit rate and with a smaller image, there may still be a discrepancy between the receipt of the audio signal and the receipt of the image, with the latter experiencing significantly greater latency than the former. This can lead to a loss of synchronisation between the displayed image and the audio discussion which relates to the displayed image.

Such a loss of synchronisation due to latency may manifest itself in various ways. For example, a person sharing their screen may be displaying and making comments about a current image, whereas other participants are still looking at a previous image while they wait to receive the current image. The other participants may therefore mistakenly consider the comments as directed to the previous image. As another example, the person sharing their screen may pan or zoom an image to view different features in the image and provide audio comments about these currently viewed features. However, the other participants may still be looking at previously display features and may mistakenly consider the comments as directed to these previous features.

It would be desirable to mitigate the above problems in an on-line conferencing system sing from the high latency associated with the transfer of large medical images and the potential resulting loss of synchronisation between an audio signal and displayed medical images.

### SUMMARY

A computing system and a method of operating system a computer system are provided. In one aspect, the computing system comprises a local store for holding at least one medical image and a display for displaying a medical image from the local store. The computing system further comprises an audio system for receiving audio input from a user of the computing system; such audio input may relate to said medical image. The computing system further comprises a graphics system for the user to perform multiple different image manipulations of said medical image using graphics commands. The computing system further comprises conferencing software to perform real-time communications with one or more other computing systems as part of an on-line conference. Performing real-time communications includes: sharing the audio input from the user with the one or more other computing systems in the on-line conference. Performing real-time communications further includes sharing the graphics commands with the one or more other computing systems to allow the one or more other computing systems to perform the different image manipulations locally as part of the on-line conference.

In another aspect, a computing system comprises a local store for holding at least one medical image received from another computing system; a display for displaying a medical image from the local store; an audio system for providing an audio output from an audio signal, wherein such audio output may relate to said medical image; and a graphics system to allow multiple different image manipulations of said medical image to be performed using graphics commands. The computing system further comprises conferencing software to perform real-time communications with one or more other computing systems as part of an on-line conference. Performing real-time communications includes receiving the audio signal from another computing system in the on-line conference; and receiving graphics commands from said another computing system to perform the different image manipulations locally as part of the on-line conference.

In some cases, a conferencing system includes the computing system of one aspect above in combination with the computing system of the another aspect above.

In another aspect, a method of operating a computing system is provided. The method includes holding at least one medical image received from another computing system in a local store of the computing system. The method further includes displaying a medical image from the local store on a display of the computing system and providing an audio output from an audio signal, wherein such audio output relates to said medical image. The method further includes performing multiple different image manipulations of said medical image using graphics commands of a graphics system and running conferencing software to perform real-time communications with one or more other computing systems as part of an on-line conference. Performing real-time communications includes: receiving the audio signal from another computing system; and receiving graphics commands from said another computing system to perform the different image manipulations locally as part of the on-line conference. In such a method, at least part of the conferencing software may run inside a browser.

The methods described herein may be implemented using a computer program comprising instructions that are executed by one or more processors in a computer system. The computer program may be stored on a non-transitory storage medium.

### BRIEF DESCRIPTION OF THE DRAWINGS

Various examples and implementations of the invention will now be described in more detail by way of example only with reference to the following drawings:

FIG. 1 is a schematic diagram showing an example configuration of users and client computers for an on-line conference as described herein.

FIG. 2 is a schematic diagram showing an example configuration of a client computer (computing system) such as shown in FIG. 1.

FIG. 3 is a schematic flowchart showing an example of a computer-implemented method for setting up and running an on-line conference involving medical images as described herein.

FIG. 4 is a further schematic flowchart showing an example of a computer-implemented method for setting up and funning an on-line conference involving medical images as described herein.

FIG. 5 is a schematic diagram showing an example of a screen for selecting images to include in an on-line conference as described herein.

FIG. 6 is a diagram showing an example of a screen during an on-line conference as described herein involving two shared images.

### DETAILED DESCRIPTION

As described herein, and by way of overview, an on-line conferencing system is provided. This conferencing system may be provided as a standalone system, or may be provided as a plug-in for an existing conferencing system. The on-line conferencing system generally supports the real-time exchange of an audio signal, typically representing the speech of the participants in the conference. The on-line conferencing system also supports the exchange of data, such as one or more medical images. In most implementations, the on-line conferencing system also supports the real-time exchange of a video signal, typically providing a view of the user at each respective client. Accordingly, the on-line conferencing system described herein may also be regarded (in most cases) as an audio and/or video conferencing system.

In operation, the on-line conferencing system is arranged to provide to all participants an initial transmission, in full, of the medical images to be included in the review (or for any other use). The provision of the medical images may be performed prior to the start of the review, or else during an initial phase of the conference. The medical images received in this manner may then be saved on each client device for use during the subsequent conference.

After the medical images have been received and stored as above, a participant may in the on-line conference may seek to discuss a medical image with the other participants. In this situation, rather than transmitting a portion (or all) of the image itself (such as by sharing an application that outputs this image), the on-line conferencing system may instead transmit reference information which specifies the location of the image portion to be shared, typically along with other relevant parameters such as the orientation and magnification of the portion of image to be shared. This reference information about the image is very compact and so can be distributed rapidly to other users with little or no latency. The other users can then use the received reference information to access their own locally stored version of the image data (as already sent to them in full detail prior to the on-line conference). Such local access can be performed quickly, since the information flow is within a single machine (rather than having to use an external network). Accordingly, this approach helps to ensure synchronisation and low latency between different participants, especially in relation to different data types, such as image, audio, video, even for high resolution images. This synchronisation (low latency) reassures participants that their audio discussions and comments are properly linked to the same referenced portion(s) of the medical image for all participants.

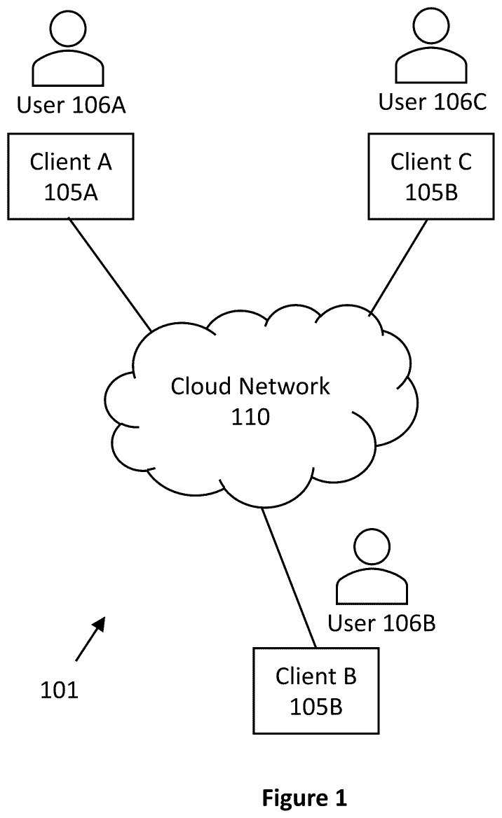

Turning now to FIG. 1, this is a schematic diagram showing an example configuration of users and client computers in an on-line conferencing system 101 as described herein. FIG. 1 depicts the conferencing system 101 as including three users 106A, 106B, 106C, each user being provided with a respective client computer system 105A, 105B, 105C. The client systems are generally standard computing devices, for example, personal computers or laptops. In some implementations, the client systems 105A, B, C may all be the same type of device, in other implementations, the client systems 105A, 105B, 105C may be different types of device. FIG. 1 depicts three users 106A, 106B, 106C (and their respective clients) as part of conferencing system 101, but it will be appreciated that the number of participants (users) in the conferencing system 101 may vary with time, and there may be more or fewer participants according to the present circumstances.

The conferencing system 101 is referred to as on-line because it is implemented by real-time communications links. The conferencing system 101 as described herein supports at least a real-time audio conferencing facility for the users 106A, 106B, 106C and also supports data exchange between the clients 105A, 105B, 105C, which may be used for sharing medical images and other data. In most cases, the conferencing system 101 will also support video conferencing, so that the participants are able to see videos of one another. These videos may be implemented in a manner to conserve bandwidth, such as by having a relatively small image size terms of number of pixels), a relatively small number of bits per pixel (limiting dynamic range for colour and tone), and/or a relatively low frame rate.

The clients 105A, 105B, 105C are shown in FIG. 1 as being linked by a cloud network 110, which may be provided in whole or in part by the Internet. In other cases, the cloud network 110 may be formed using a private network (intranet). The clients 105A, 105B, 105C may connect to the cloud network 110 using any suitable facility, such as a wi-fi access point (not shown in FIG. 1). In some implementations, the cloud network 110 may include one or more servers for hosting the conference. In this case, the communications between the client systems 105A, 105B, 105C may be handled in a centralised manner, with transmissions from a client system (say client 105A) being directed to the server, and the server then forwarding the transmissions to all the other clients, namely clients 105B and 105C in the particular example of FIG. 1; transmissions from clients 105B and 105C would be handled in an analogous manner. In an alternative approach, the cloud network 110 does not include such a server, and the conference is managed in a less centralised, more distributed manner. In other words, client 105 would make separate transmissions to all the other clients, namely clients 105B and 105C in FIG. 1, and transmissions from clients 105B and 105C would be handled in a corresponding manner.

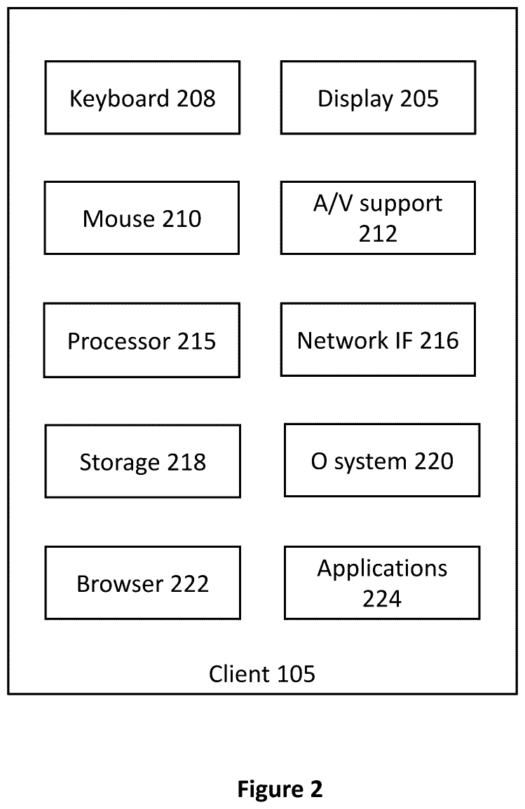

FIG. 2 is a schematic diagram showing an example configuration of a client device 105, such as may be used in the conferencing system 101 and as shown in FIG. 1. The client includes one or more processors 215 for executing computer programs and at least one form of network interface 216 for communicating with external devices (such as the cloud network 110 and other client devices as shown in FIG. 1). In practice a client device 105 may support connectivity via a range of wired and wireless network interfaces. For example, client 105 may support one or more wireless interfaces, such as wi-fi, Bluetooth, and/or near field communications (NFC), and/or one or more wired interfaces, such as USB and Ethernet.

The client device 105 of FIG. 2 further includes a display 205 and a keyboard 208 for output and input respectively. Note that in some devices, the keyboard 208 may be implemented using a touchscreen on display 205. The client device 105 may further include a mouse or similar input device, such as for controlling a cursor on the display 205 and/or for making selections and providing other commands to the client device 105. The client device further includes support 212 for audio/visual (AV) media, such as may be provided by a microphone for audio capture and a speaker for audio output. The microphone and/or speaker may be integrated into the client device 105, or may be provided by ancillary devices.

The client device 105 further includes storage 218, which is usually provided as a combination of volatile memory (such as dynamic RAM) and non-volatile storage (such as a hard disk drive or solid state ROM). The non-volatile storage is generally used to retain programs and data. If a program is to be executed on processor 215, then the program instructions are generally loaded from storage into volatile memory for faster access. Data for use by the program which is being executed may likewise be loaded into volatile memory for faster access by the program. In some cases, programs may be downloaded into memory for execution by processor 215 (without first being stored in non-volatile storage). The may client 105 further include software (computer programs) for controlling and utilising the client. In particular, the client device 105 of FIG. 2 includes an operating system 220, a browser 222 such as Edge, Firefox or Chrome, and one or more application programs 224.

In some implementations of the approach described herein, the web browser 222 is used to download application software for participating in the online conference from a server (which may be located, for example, in the cloud network 110 of FIG. 1). This software may be written using Javascript and/or WebGL, both of which may be run directly from within the browser. This allows the software to be utilised by any computing device which includes a standard web browser 222, without having to install any additional software locally on client 105. It will be appreciated however that many other implementations are possible, including the use of a local application 224 to perform the functionality described herein, and/or the use of different languages or interfaces (other than Javascript or WebGL) according to the circumstances of any given implementation.

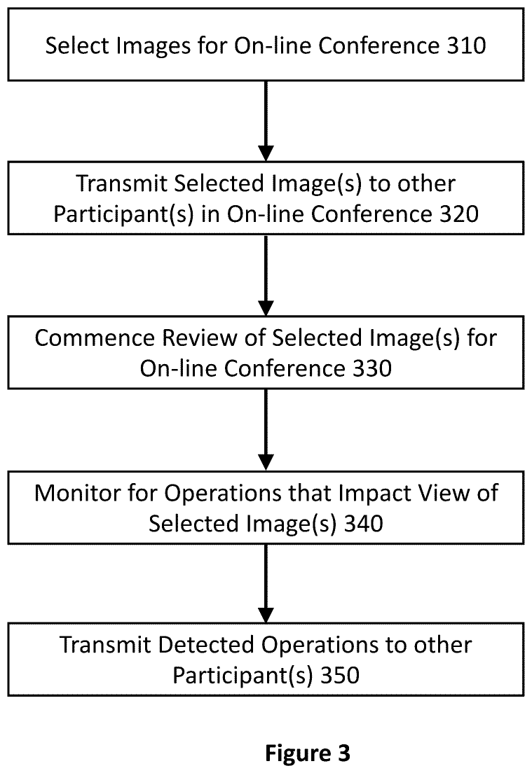

FIG. 3 is a schematic flowchart showing an example of a computer-implemented method for setting up and running an on-line conference involving medical images as described herein. In particular, for present purposes, we assume that user A 106A is setting up and participating in an on-line conference with users 106B, 106C (and their respective client systems 105B, 105C), and FIG. 3 illustrates the operations performed by user A 106A for setting up and participating in such an on-line conference.

In operation 310, the user A 106A selects one or more medical mages for submission to the on-line conference for review with users 106B, 106C during the on-line conference. This selection may be made by user A 106A prior to the start of the on-line conference or during an initial phase of the on-line conference. A further possibility is that an image is selected during the conference itself, for example, because the need or value of including the image in the on-line conference only becomes apparent during the on-line conference itself. It will be appreciated that a given on-line conference may involve multiple images, and these may not all be selected at the same time. For example, user A 106A may make an initial selection of one or more images and transmit them prior to the on-line conference and/or during an initial phase of the on-line conference. During the on-line conference, it may be decided to review one or more further images, not part of the original selection. For example, the further images might relate to a different imaging modality, or represent an earlier image which pre-dates the initially selected images to establish a longer time-line of the medical condition for review.

In some cases, the selected image(s) may already be available on client system 105A for direct review and selection by user A 106A, for example because user A 106A is already working on one or more of these images. The user A 106A may implement a local store for such images within storage 218. In other cases, one or more of the images may be held in a (remote) patient or image database, or across multiple such databases. The client system 105A may use the network interface 216 and cloud network 130 to access the image database(s) (not shown in FIG. 1) for selecting and downloading the desired images for the review. Access to the image database may require user A 106A to provide some form of authentication or authorisation to protect the confidentiality of patient images held within the database(s).

Once the user A 106A has selected one or more images to include in the review, these selected images are downloaded (as required) from the image database(s) to client device 105A. The medical images may be encrypted and/or password protected, again to support the confidentiality of the patient images. It will be appreciated that the storage of medical images in such an image database for on-line access and downloading by a clinician is known (per se) in some existing systems.

At operation 320, the selected images which have been downloaded to client device 105A are provided to the other participant(s) in the on-line conference. As noted above, for convenience of explanation, we assume that the other participants correspond to users 106B, 106C and their respective client devices 105B, 105C as shown in FIG. 1. There are a number of ways in which the selected images may be provided to the other participants. By way of example, the user A 106A may email the images to the other users, or may put the images into some form of shared folder from which the images may be downloaded to the relevant client devices 105B, 105C. In this latter option, user A 106A may provide users 105B, 105C with a link to the shared folder (if they do not already have such a link, such as from previous collaborative activity). A further possibility is that rather than directly transmitting any images from the client device 105A to the client devices 105B, 105C, the user A 106A may instead send a link or reference to the images to be shared, for example, based on a file name, URL or other identifier. This link or reference may then allow the users 106B, 106C to access and download the selected image(s) to client devices 105B, 105C, typically from the same image database that was used by user A 106A to access and download the selected image(s) to client device 105A (or from any other suitable image database). One advantage of this latter technique is that users 106B, 106C may likewise provide their own authentication or authorisation to access the selected image(s) from the image database. This would then provide more security for the images compared with (say) user A 106A directly emailing the images to users 106B, 106C.

It will be appreciated that the images may be provided in different ways to different users. For example, once user A 106A has selected an image to include in an on-line conference, user A 106A may email the downloaded image to user B 106B, but send a link or reference for the image to user C 106C to allow user C 106C to download the selected image from an image database. (This approach might be appropriate for example if user A 106A and user C 106C both have access to the image database, but user B 106B does not have such access, perhaps because they are affiliated to a different institution).

If the on-line conference incorporates multiple images, the different ages may be provided by user 106A to the other users 106B, 106C in different ways. For example, once user A 106A has selected first and second images to include in an on-line conference, user A 106A may email the downloaded first image to users 106B, 106C, but send a link or reference for the second image to users 106B, 106C to allow users 106B, 106C to download the selected second image from an image database. (This approach might be appropriate for example if the first and second images have different modalities, and images having the second modality are stored in an image database that provides access to people with suitable authorisation, whereas images having the first modality are not entered into such an image database).

It will be appreciated that other factors may affect the way in which user A 106A may provide one or more images to other participants in the on-line conference. For example, this may be impacted by the timing of providing the image (such as before the conference or during the conference), the network facilities and bandwidth available to different users at different times, and so on. Irrespective of how this image transmission to users 106B, 106C is accomplished, users 106B, 106C save the image(s) provided by user A 106A into some form of local store on client devices 105B, 105C respectively, for example as provided by storage 218 for subsequent use of the image(s) during the on-line conference. In some cases the selected images may be stored in encrypted form (or with some form of password protection). The images may then be decrypted (and/or a password provided) when the images are incorporated into the on-line conference.

At operation 330, the review of the selected images during the on-line conference is now initiated. As noted above, additional images may potentially be provided during the on-line conference, however, this may be associated with some delay. Accordingly, if possible it is helpful for the selected images to be provided to the users 106B, 106C prior to the on-line conference, or during an initial phase of the conference, for example while the participants are discussing preliminary matters that do not depend on access to the images. Operation 330 may also involve initiating a software application 224 to perform the processing described herein. Thus selecting (and potentially downloading) an image and then transmitting the image from client device 105A to the other clients 105B, 105C may, if so desired, be implemented by conventional existing software such as a web browser 222 and an email system. However, the operation of the on-line conferencing as further described below in relation to operations 340 and 350 relies on an additional software application 224 which goes beyond such conventional existing software. As mentioned above, this additional software application 224 may execute within a browser 222 to ensure widespread support on many computer systems without having to specifically install local software on client device 105A. Accordingly, the software application 224 may, for example, open the selected medical images stored in the local store so that they are ready for use by the software application during the on-line conference.

During the on-line conference, the software application 224 on the client device 105A performs operation 340 to monitor for operations by user A 106A that relate to the display and manipulation of the selected medical images. By way of example, we assume that user A 106A opens an image during the on-line conference which has previously been provided to the other users 106B, 106C (such as at operation 320). The software on client device 105A (such as application 224) may provide a facility for the user A 106A to open the image. Furthermore, in response to the user A 106A opening this image, the software on client 105A may transmit a message to the other participants, clients 105B, 105C, as per operation 350, providing a notification that user A 106A has opened a selected (identified) image on client device 105A. In response to this notification, the software on client device 105B opens the identified image on client device 105B, and likewise the software on client device 105C opens the identified image on client device 105C, both client devices 105B and 105C having previously been provided with the identified image at operation 320. In some cases, the software on clients 105B, 105C may open the identified image automatically; in other cases, the software may seek input from the relevant user 106B, 106C to confirm that the identified image(s) should be opened. (If a user does not provide such confirmation, then this user would not be able to use or see the identified image during the on-line conference).

For present purposes (but without limitation), we now assume that user A 106A acts as a host for at least part of the review of the selected image. The role of host enables user A 106A to perform various operations with respect to the selected image. The exact set of available operations (image manipulations) may vary from one implementation to another, but by way of example, the operations may include panning across the image, zooming into a particular portion of the image, changing the contrast of the image, rotating the image, and changing the view direction of a 3D image. In particular, the user A 106A acting as host may perform such manipulations during the review to facilitate better understanding of the images by the users participating in the call.

The above image manipulations generally involve the software (application 224) receiving input from user A 106A, for example via mouse 210 in conjunction with display 205, to instruct such an image manipulation. For example, panning across an image might be instructed by a user holding down the right mouse button (select) and then moving the mouse to instruct dragging the image across the display 205. The software may convert the user input into a graphics command to send to the operating system or other low-level code (e.g specialist graphics code) to implement the user input on the display 205. For example, the user input may be converted into one or more WebGL commands (see https://www.khronos.org/webgl/) for execution by an WebGL implementation to perform the desired update of display 205 as requested by the user input. According to the present approach, the graphics commands are not only passed onto the appropriate graphics implementation on the host client device 105A, but are also transmitted to the other client devices 105B, 105C participating in the on-line conference (as per operation 350) for local execution by these other participants.

In this approach, after the initial provision of the image at operation 320 to other participants, subsequent communications are performed with respect to graphical commands as per operation 350, without the further transmission of any images or parts thereof. For example, such a graphical command may indicate an image on which the command is to be implemented (or this may default to a currently active image or window). The graphical commands then define the nature of the image manipulation requested by the user, together with any associated parameters. For example, in the above example of a pan operation, the graphical command may specify “pan” as the image manipulation to be performed, and further specify a direction and distance of the panning operation (or equivalently, specify a new location which is the target of the panning operation).

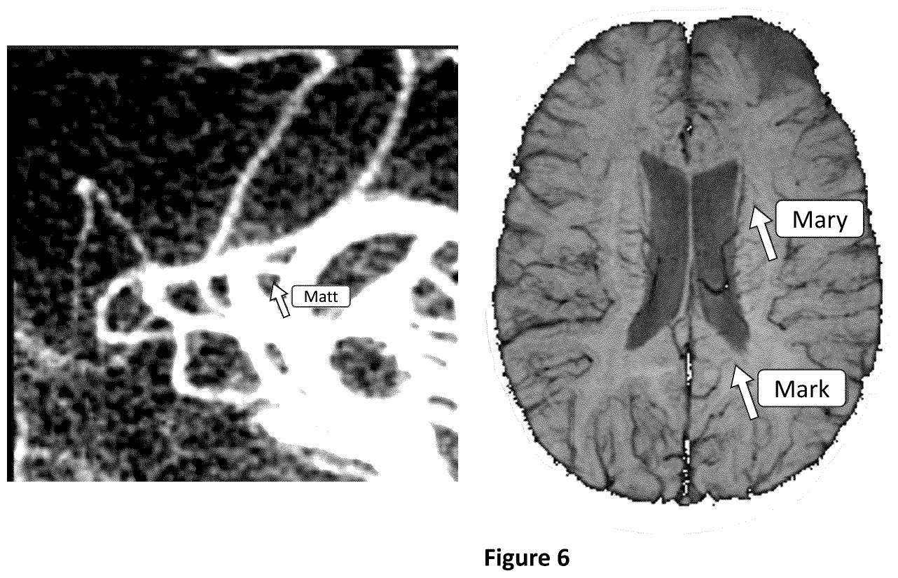

It will be appreciated that such graphics commands may therefore be implemented using relatively short strings of characters, and the transmission of such characters can be performed with very little or no delay (latency) between the host system (e.g. client device 105A) implementing the user specified image manipulation and the same image manipulation then being performed by the other participants in the on-line conference (client devices 105B, 105C). In some implementations, the on-line conferencing software may transmit the graphics commands and audio signal in the same composite packets, thereby ensuring that the image updates and any audio commentary are maintained in synchronism with one another. In other implementations, there may be respective packets for transmitting the graphics commands and audio signal, in which case the synchronism is based on the small packet sizing, and hence the low latency for both the audio signal and the graphics commands. It will be appreciated that generally it is only the host who is sending both graphics commands and an audio signal to the other users in the on-line conference, since users who are not the host may only send their audio signal to other users (not any image updates). However, such other users may send certain types of graphics commands, for example indicating cursor position (as discussed below with respect to FIG. 6). It is also noted that some on-line conferencing systems might not support or require a formal host to be identified. In this latter approach, the participants may, for example, use the audio channel to agree who is going to perform the next image manipulation. A further possibility is that the participants may change the identity of the host as the on-line conference progresses.

The on-line conference now typically cycles between operations 340 and 350. In particular, at operation 340 a command for an image manipulation is detected. At operation 350, this command is implemented locally on client device 105A, but also transmitted from client device 105A to the other client devices 105B, 105C for implementation on client devices 105B, 105C. The processing of client device 105A then returns to operation 340 to wait for and detect the next command for image manipulation. At operation 350, the software again extracts the graphical commands associated with this image manipulation and transmits these graphical commands to client device 105B, thereby again allowing the desired graphical commands to be implemented both on client device 105A (where the graphical commands were entered by user A 106A) and also on clients 105B and 105C, so that users 106B, 106C have the same graphical commands implemented on client devices 105B, 105C as on client device 105A. This then allows the client devices 105B, 105C to track and mirror the successive image manipulations performed by user A 106A for presentation to users 106B, 106C. This cycling of operations 340 and 350 may continue until the end of the on-line conference.

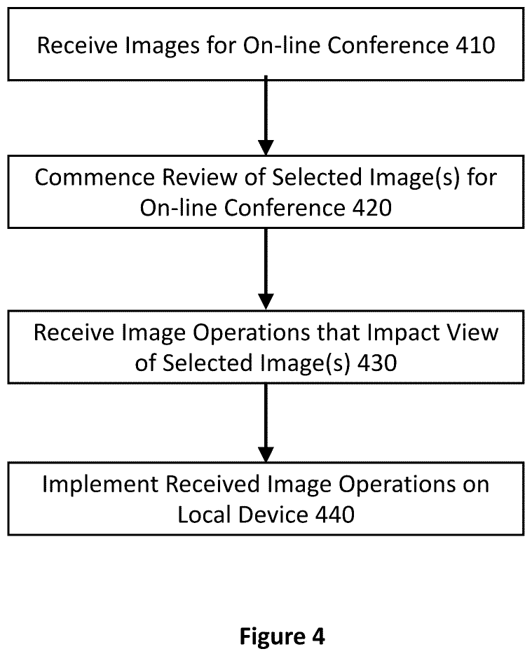

FIG. 4 is a further schematic flowchart showing an example of a computer-implemented method for setting up and running an on-line conference involving medical images as described herein. FIG. 4 can be considered as complementary to FIG. 3, and again we assume that user A 106A is setting up and participating in an on-line conference with users 106B, 106C (and their respective client systems 105B, 105C), FIG. 4 illustrates the options performed by users 106B, 106C for setting up and participating in such an on-line conference. In particular, for present purposes we describe the involvement of user B 106B as the other participant in the on-line conference and it will be appreciated that user C 106C will generally have the same or an analogous involvement.

FIG. 4 commences with operation 410 in which one or more selected images are received by client device 105B from client device 105A (representing the host of the conference). Thus operation 410 of FIG. 4 can be regarded as complementary to operation 320 of FIG. 3. As discussed above, client device 105B may receive the actual image(s) from client device 105, or alternatively some form of link or reference to allow the selected image(s) to be provided or downloaded to client device 105B. Once the selected image(s) have been obtained by the client device 105B, they may be held within a local store which may be implemented using storage 218. It will be appreciated that this local storage allows future access to the selected images stored on client device 105B to be performed quickly in real-time, without (for example) any delay that might be experienced when trying to access a remotely stored image. In some implementations, the client device 105B may send a confirmation back to the host client device 105A that the selected images are now locally stored on client device 106B. If no such confirmation is received by client device 105B, then this may potentially delay beginning the review at operation 330, for example, if such selected images are required as part of the review. In these circumstances, the client device 105A may make another attempt to provide the selected images (or links thereto) to the other participants such as client 105B.

At operation 420, client device 105B begins the review of the selected images as part of the on-line conference. It will be appreciated that operation 420 of FIG. 4 is generally complementary to operation 330 of FIG. 3, and as discussed above, beginning the review may involved launching suitable software, such as application 224, on the client device 105B. As with the corresponding software application on client device 105A, the software utilised by client device 105B may be run within a browser 222 to ensure a high level of compatibility with many different computer systems.

As part of starting the review, client 105B may open the selected images, potentially upon receipt of an instruction from client 105A to do so. The client 105B may confirm back to client 105A when the selected images are open on client 105B. At this stage, a selected image displayed on the client 105B should correspond directly with a selected image displayed on client 105A; in other words, the two displays 205 for clients 105A and 105B should be in synchronisation with one another.

In some implementations, starting the review and launching the software in operation 420 results in a screen that lists the images available in the local store for client device 105B. A user can select one of these images, or a portion of such an image (e.g. a slice if the image is 3D) for display on display device 205.

At operation 430, the client device 105B receives the image operations transmitted by client device 105A in operation 350 in FIG. 3. As explained above. these image operations (manipulations) may be received as a set of one or more graphics commands, for example, according to an WebGL interface. At operation 440, these graphics commands are passed to the graphics engine (e.g. an WebGL, implementation) for execution on client device 105B. This results in the display for client device 105B tracking the display for client device 105A, since both receive and process the same sequence of graphics commands to produce matching display outputs.

In the above description, we have assumed that client device 105A both distributes the images prior to the on-line conference to review the images, and then hosts the on-line conference itself (whereby the graphics commands all originate from the host). However, other implementations and/or conferences may have a different approach. For example, client device 105B may distribute the image(s) to clients 105A, 105C prior to the on-line conference. Then, in the conference itself, the users 106A, 106B, 106C may agree that client device 105C should be the host for the on-line conference. Furthermore, the identity of the host may vary during the on-line conference. For example, user B 106B may be host at the beginning when presenting the thoughts of user B 106B. Subsequently, user C 1060 may become host (by agreement with users 106A, 106B), for example to allow the new host 106C to present his/her thoughts on a patient under review.

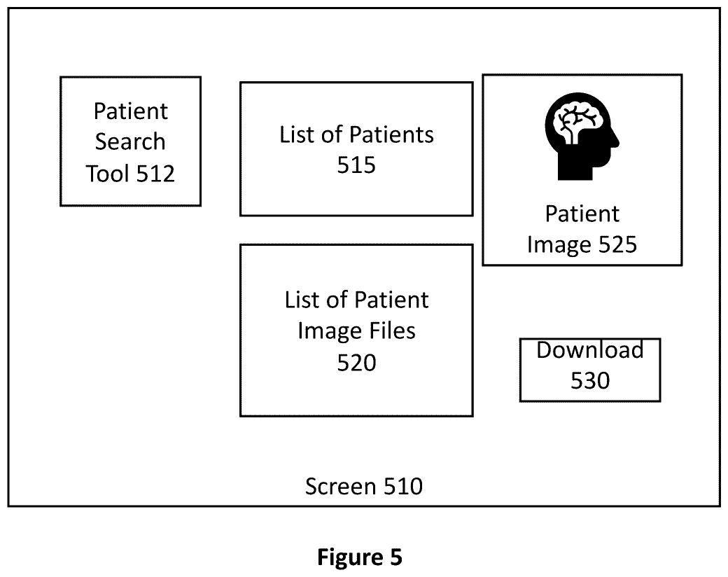

FIG. 5 is a schematic diagram showing an example of a screen 510 as displayed by an application program 224 on a display unit 205 for selecting one or more images to download for use in an on-line conference. This screen of FIG. 5 may be provided from a URL supported by a server (not shown) in the cloud network 110. A user of the on-line conferencing system 101 therefore may access this URL, to obtain the screen 510 shown in FIG. 5. Note that prior to obtaining access to the screen 510, the user 106A, B, C may have to provide appropriate authorisation (authentication) details, such as a password.

Once the authorisation (authentication) has been completed to provide access to screen 510 a box 512 is displayed in the top left portion of this screen 510. This box 512 represents a search tool that allows a user to search for and identify a particular patient or subject. For example, the search might be made with respect to parameters such as patient name, patient date of birth, and hospital number for the patient.

In response to the patient search, a list of patients 515 that fit the search criteria may be displayed on screen 510. A user is then able to select a particular patient from the list 515, and then a list of image files associated with this particular patient may be displayed in list 520. The list 520 may provide brief information about each image, such as the date created and the imaging modality. If an image is selected from this list 520, then the image may be displayed in box 525. In addition, a user can then select one or more of the images from list 520 to save into their local store (implemented in storage 218), for example by activating the download button 530.

FIG. 6 is a diagram showing an example of a screen during an on-line conference as described herein involving the display of two shared images (as supported by the on line conferencing software). Each shared image may be controlled as described above (in effect independently of the other shared image). Note that FIG. 6 does not represent the complete screen displayed to a user, which may include other features like control facilities, and small video feeds of the other participants in the conference (such as commonly provided by Zoom, Teams and other video-conferencing platforms).

In this screen the two shared images may have different hosts. For example, if we assume that FIG. 6 corresponds to the screen view for user A 106A on client device 105A, then client device 105A might be the host for the left-hand image in FIG. 6 but the host for the right-hand image in FIG. 6 might be different, e.g. client device 105B on behalf of user B 106B. This split of hosting may be beneficial, for example, if the two images were acquired with first and second imaging modalities, and the left image is hosted by a clinician who is an expert in the first imaging modality and the right image is hosted by a clinician who is an expert in the second imaging modality.

FIG. 6 also shows cursor positions for users in the conference, for example, the left-hand image has a displayed cursor for Matt (corresponding say to user A 106A), while the right-hand image has display cursors for Mary and Mark (corresponding to say users B, C 106B, 106C). The cursor positions can be shared using a similar approach to that described with reference to FIGS. 3 and 4. However, unlike for the medical images themselves, there may be no host in the on-line conference who is responsible for manipulations of the displayed image(s). This is because each user has their own cursor which can be positioned using graphics commands entered by that user. The graphics commands can then be detected and shared with the other client devices 105. The on-line conferencing system can control the cursors from different users so that they look different from one another—e.g. by having different colours and also the name of the user 106A, B, C responsible for each respective cursor. Note that the displayed cursors from users other than the host of a given image are generally available for display rather than control. In particular, such a cursor for a user who is not the host may be moved around the screen, but cannot be used to perform any command such as an image manipulation on the screen.

The cursors shown in FIG. 6 may be considered as a form of annotation, in that they are not part of the displayed medical images per se, but are superimposed on the medical images. The on-line conferencing system may provide support for other forms of annotation, such as users 106A, 106E, 106C drawing lines or arrows which are superimposed on the displayed medical image. Such annotations may assist in the review, for example, they may be used to draw attention to particular features in the shared image.

Accordingly, an on-line (real-time) conferencing system has been described to support the sharing and review of medical images by multiple clinicians. The on-line conferencing system is implemented typically on standard hardware such as personal computers, laptops and so on. In addition, the conferencing software may be provided for execution within a browser. Therefore the on-line conferencing system has wide accessibility to users, since there is no need for specialised hardware or for the installation of specialised conferencing software,

Prior to the on-line conference, the medical images for review are distributed to other participants in the video conference for local storage of the images by each participant. Such distribution may, for example, be performed by using email to send the images, or by sending links to the other participants so they can download their own copy of the images. In some cases, it may be advantageous for reasons of confidentiality (security) to use downloads from a server rather than transmission using email over a public network connection.

During the on-line conference, a reference to an image or to a portion of the image is transferred to other participants (rather than transmitting the image itself or a portion of the image, such as by screen sharing). For example, such a reference might be provided by defining an image portion which has a lower left corner at row 256 and column 32, a height of 128 rows, and a width of 64 columns. Such a reference may be provided, for example, as part of a graphics command the performs some form of image manipulation involving this portion of the image. Each participant receives the reference information and uses it to process the specified image portion from its own locally stored version of the image for. It will be appreciated that sending such a reference or graphics command during the on-line conference, rather than the image data itself, involves far less bandwidth, and so avoids or greatly reduces latency during the on-line conference. This helps to ensure that different aspects of the conference, such as (i) audio, and (ii) display of a medical image, remain in proper synchronisation with one another.

The approach described herein has a number of benefits with respect to an existing approach of including images by application sharing in a video conferencing system. By providing the participants instead with medical images in advance of an on-line conference, the conference itself does not experience delays (latency) associated with the large bandwidth required for transmitting the medical images. This then helps to improve synchronisation between the display of a medical image and other components of the on-line conference, such as audio discussions of the medical image. Furthermore, in this approach, the images are only transmitted once, prior to the video conference. In contrast, with application sharing, an image might be repeatedly displayed, interspersed with the display of other images or outputs. In some implementations, the shared image may need to be re-transmitted each time it is redisplayed, thereby imposing further network delays. Also, because image data is not transmitted during the on-line conference itself, this provides protection against any security weakness in the conferencing platform (which might potentially lead to unintended access to patient images).

The present disclosure further provides a method and system for providing on-line conferences relating to medical images. Such conferences are primarily described herein as being used for review meetings, but may also be used for other purposes, such as helping to perform telemedicine. Also provided herein is a computer program comprising program instructions that when executed on one or more computer systems may cause the computing system(s) to perform such a method. The computer program may be provided on a suitable storage medium such as described below. The system described herein may be implemented using a combination of hardware and software. The hardware may comprise one or more computing systems, which may be general purpose computers. In some implementations, the hardware may include more specialised components, such as a graphical processing unit (GPU) and so on to facilitate processing of images. The software generally comprises one or more computer programs to run on the hardware. These computer programs comprise program instructions which are typically loaded into memory of the computing system(s) for execution by one or more processors to cause the computing system(s) to provide on-line conferencing as described herein. The computer program (s) may be stored in a non-transitory medium prior to loading into memory, for example, on flash memory, a hard disk drive, etc. The operations of the computer system(s) may be performed sequentially and/or in parallel as appropriate for any given implementation.

Various implementations and examples have been disclosed herein. It will be appreciated that these implementations and examples are not intended to be exhaustive, and the skilled person will be aware of many potential variations and modifications of these implementations and examples that fall within the scope of the present disclosure. It will also be understood that features of particular implementations and examples can typically be incorporated into other implementations and examples (unless the context clearly indicates to the contrary). In summary, the various implementations and examples herein are disclosed by way of illustration rather than limitation, and the scope of the present invention is defined by the appended claims.

We claim:

1. A computing system comprising:
a local store for containing at least one medical image;
a display for displaying a medical image from the local store;
an audio system for receiving audio input from a user of the computing system, wherein such audio input may relate to said medical image;
a graphics system to allow the user to perform multiple different image manipulations of said medical image using graphics commands;
conferencing software to perform real-time communications with one or more other computing systems as part of an on-line conference, wherein performing real-time communications includes:
sharing the audio input from the user with the one or more other computing systems; and
sharing the graphics commands with the one or more other computing systems to allow the one or more other computing systems to perform the different image manipulations locally as part of the on-line conference.

2. The computing system of claim 1, wherein the graphics commands and audio input are shared in synchronisation with one another.

3. The computing system of claim 1, wherein the computing system is configured to transmit a copy of said medical image to the one or more other computing systems prior to using the conference software to perform real-time communications with the one or more other computing systems as part of the on-line conference.

4. The computing system of claim 1, wherein the computing system is configured to transmit a link or reference to said medical image to the one or more other computing systems prior to using the conference software to perform real-time communications with the one or more other computing systems as part of the on-line conference.

5. The computing system of claim 1, wherein sharing the graphics commands comprises intercepting the graphics commands directed to the graphics system and forwarding the intercepted graphics commands to the one or more other computing systems.

6. The computing system of claim 1, wherein the graphics commands identify a medical image on which the image manipulation is to be performed, the type of image manipulation, and optionally one or more parameters relating to the image manipulation.

7. The computing system of claim 1, wherein performing real-time communications further includes:
sharing a mouse or cursor position as controlled by the user with the one or more other computing systems.

8. The computing system of claim 1, wherein at least part of the conferencing software runs inside a browser.

9. A method of operating a computing system comprising:
holding at least one medical image in a local store of the computing system;
displaying a medical image from the local store on a display of the computing system;
receiving audio input from a user of the computing system, wherein such audio input relates to said medical image;
the user performing multiple different image manipulations of said medical image using graphics commands of a graphics system;
running conferencing software to perform real-time communications with one or more other computing systems as part of an on-line conference, wherein performing real-time communications includes:
sharing the audio input from the user with the one or more other computing systems; and
sharing the graphics commands with the one or more other computing systems to allow the one or more other computing systems to perform the different image manipulations locally as part of the on-line conference.

10. The method of claim 9, wherein the graphics commands and audio input are shared in synchronisation with one another.

11. The method of claim 9, wherein the computing system is configured to transmit a copy of said medical image to the one or more other computing systems prior to using the conference software to perform real-time communications with the one or more other computing systems as part of the on-line conference.

12. The method of claim 9, wherein the computing system is configured to transmit a link or reference to said medical image to the one or more other computing systems prior to using the conference software to perform real-time communications with the one or more other computing systems as part of the on-line conference.

13. The method of claim 9, wherein sharing the graphics commands comprises intercepting the graphics commands directed to the graphics system and forwarding the intercepted graphics commands to the one or more other computing systems.

14. The method of claim 13, wherein the graphics commands identify a medical image on which the image manipulation is to be performed, the type of image manipulation, and optionally one or more parameters relating to the image manipulation.

15. The method of claim 9, wherein performing real-time communications further includes:
sharing a mouse or cursor position as controlled by the user with the one or more other computing systems.

16. The method of claim 9, wherein at least part of the conferencing software runs inside a browser.

17. A computing system comprising:
a local store for containing at least one medical image received from another computing system;
a display for displaying a medical image from the local store;
an audio system for providing an audio output from an audio signal, wherein such audio output may relate to said medical image;
a graphics system to allow multiple different image manipulations of said medical image to be performed using graphics commands;
conferencing software to perform real-time communications with one or more other computing systems as part of an on-line conference, wherein performing real-time communications includes:
receiving the audio signal from another computing system; and
receiving graphics commands from said another computing system to perform the different image manipulations locally as part of the on-line conference.

18. The computing system of claim 17, wherein the received graphics commands and the received audio signal are in synchronisation with one another.

19. The computing system of claim 17, wherein the at least one medical image received from a server is downloaded over a secure connection with said server.

20. The computing system of claim 17, wherein the graphics commands identify a medical image on which the image manipulation is to be performed, the type of image manipulation, and optionally one or more parameters relating to the image manipulation.
